(19) United States
(12) Patent Application Publication
(10) Pub. No.: US 2026/0325771 A1
(43) Pub. Date: Oct. 22, 2026

(54) APPARATUSES, SYSTEMS, AND METHODS FOR TRANSMITTING THERAPEUTIC GAS

(71) Applicant: Nanjing Novlead Biotechnology Co., Ltd., Nanjing, Jiangsu (CN)

(72) Inventors: Yuyan ZHANG, Nanjing, Jiangsu (CN); Wangkun ZHANG, Nanjing, Jiangsu (CN); Xianghai KONG, Nanjing, Jiangsu (CN); Yuan LI, Nanjing, Jiangsu (CN); Fei WANG, Nanjing, Jiangsu (CN)

(21) Appl. No.: 19/490,319

(22) PCT Filed: Jun. 5, 2024
(86) PCT No.: PCT/CN2024/097474

§ 371 Date: Dec. 4, 2025

(30) Foreign Application Priority Data
Jun. 6, 2023 (CN) 202310660438.1
Apr. 12, 2024 (CN) 202410444278.1

(51) Int. Cl. A61M 16/00; A61M 16/12; A61M 16/20

(52) U.S. Cl. CPC A61M 16/022; A61M 16/0003; A61M 16/0057; A61M 16/125; A61M 16/203; A61M 16/209; A61M 2016/0027; A61M 2202/0007; A61M 2202/0225; A61M 2202/0275; A61M 2205/3327; A61M 2205/3334; A61M 2205/3341; A61M 2205/75; A61M 2205/8218

(57) ABSTRACT

This disclosure describes a therapeutic gas delivery device. The therapeutic gas delivery device may include a therapeutic gas source configured to generate therapeutic gas, and a gas storage section connected downstream of the therapeutic gas source. The gas storage section being configured to store at least part of the therapeutic gas from the therapeutic gas source. Furthermore, the device may include a gas output section connected downstream of the gas storage section, the gas output section being configured to output the therapeutic gas on demand. In addition, the device may include a replenishment section connected to the gas storage section, the replenishment section configured to supplement gas to the gas storage section. Moreover, the device may include a pressure control unit connected to the gas storage section, configured to stabilize a pressure inside the gas storage section. Also, the device may include a flow control unit coupled to the gas output section, configured to control a quantity of the therapeutic gas delivered through the gas output section.

### CROSS-REFERENCE TO RELATED APPLICATIONS

This application claims the benefit of priority of Chinese Provisional Patent Application No. 2023106604381, filed Jun. 6, 2023, all of which is incorporated herein by reference in its entirety.

### TECHNICAL FIELD

This present disclosure relates to the field of medical device technology, particularly to a therapeutic gas transmission system that delivers the therapeutic gas on demand.

### BACKGROUND

Inhalation therapy involves supplying therapeutic gases to patients through devices such as ventilators to achieve therapeutic effects. Taking nitric oxide (NO) gas as an example, nitric oxide has been found in recent years to play a role in transmitting important signals and regulating cell functions in the human body. It can help promote blood circulation within the body. Nitric oxide can rapidly diffuse through biological membranes without any intermediary mechanisms, transmitting information produced by one cell to the surrounding cells. Nitric oxide has various biological functions, readily participating in electron transfer reactions and the body's oxidation-reduction processes. Direct inhalation of nitric oxide therapy, approved by the United States Food and Drug Administration as a treatment for persistent pulmonary hypertension in newborns, has been proven to improve the body's oxygenation capacity and reduce the need for high-risk extracorporeal life support in critically ill patients. The administration of nitric oxide in controlled and appropriate amounts can specifically reduce pulmonary hypertension and improve oxygenation. Currently, nitric oxide inhalation therapy is widely used in neonatal respiratory medicine and is also applicable in critical care, cardiothoracic surgery, respiratory internal medicine, anesthesiology, and other clinical medical fields.

Therapeutic gases like nitric oxide are usually used in conjunction with respiratory equipment such as ventilators and anesthesia machines. The gases are transmitted to the inhalation pipeline of these devices and then inhaled by the patients for gas inhalation therapy. Breath-following therapeutic gas transmission is a preferred method of delivering therapeutic gases, as it involves transmitting the therapeutic gases in accordance with the patient's breath flow rate (i.e., the delivery of therapeutic gases is synchronized with the patient's breathing patterns). Compared to traditional methods of therapeutic gas delivery, the breath-following approach maintains a stable concentration of inhaled therapeutic gases throughout the breathing process and significantly reduces the impact of changes in respiratory mode and parameters on the concentration of inhaled therapeutic gases.

Due to the constant and significant fluctuations in breath flow rate throughout the patient's breathing process, achieving breath-following often requires rapidly transmitting a specific volume of therapeutic gas in a short time. For devices that instantly generate therapeutic gases, it is challenging to ensure the instantaneously produced therapeutic gas matches the instantaneously required transmission consumption, posing a significant challenge for instant therapeutic gas generation devices to implement breath-following transmission functionality.

The therapeutic gas transmission apparatus and system described herein allow for perfect compatibility with ventilators from different manufacturers, different ventilator modes, and even with anesthesia machines and extracorporeal membrane oxygenation (ECMO) units. They also meet the accuracy and stability needs of therapeutic gas concentration for different populations, including adults, children, and newborns with varying tidal volumes and respiratory rates, especially in high-frequency oscillatory ventilation.

Some existing instant generation devices are designed to achieve breath-following transmission functionality. As disclosed in PCT patent publication number WO2022127902A, a “pressure vessel” is placed downstream of the therapeutic gas generation device and upstream of the therapeutic gas transmission pipeline. This pressure vessel, a large-capacity gas storage tank capable of withstanding certain pressures, stores the excess generated therapeutic gas and relies on the pressure generated by the accumulated therapeutic gas inside the tank. Thus, when there is a need to rapidly output a large volume of therapeutic gas in a short time, this pressure can be relied upon to output the stored therapeutic gas from the tank, compensating for the part of the consumption that the instant preparation could not produce in time.

This vessel-based approach has disadvantages, such as pressure fluctuations inside the storage tank when a large flow rate of therapeutic gas is required from the tank, which can affect the flow rate of the output therapeutic gas and ultimately cause deviations in the concentration of the transmitted therapeutic gas. The larger the capacity of the storage tank, the better the effect of avoiding pressure fluctuations during large flow rate outputs, requiring the storage tank to be very large to ensure the absence of the aforementioned defects under some conventional flow demands. This greatly limits the miniaturization of the equipment and restricts the applicable scenarios for instant therapeutic gas generation devices. Another disadvantage is that when the storage tank's capacity is very large, therapeutic gases can accumulate inside for a long period. Taking NO as an example, NO can easily oxidize into toxic NO2, and instantly prepared NO staying too long in a large-capacity storage tank can lead to an increase in NO2 content, thereby affecting the quality of the output therapeutic gas.

### SUMMARY

According to some embodiments of the present disclosure, an apparatus for generating and/or delivering therapeutic gas (e.g., nitric oxide (NO) is provided.

In one general aspect, the therapeutic gas delivery apparatus may include a therapeutic gas source (1) configured to generate therapeutic gas. The therapeutic gas delivery apparatus may also include a gas storage section (2) connected downstream of the therapeutic gas source (1), the gas storage section (2) being configured to store at least part of the therapeutic gas from the therapeutic gas source (1). The therapeutic gas delivery apparatus may furthermore include a gas output section connected downstream of the gas storage section (2), the gas output section being configured to output the therapeutic gas on demand. The therapeutic gas delivery apparatus may in addition include a replenishment section (4) connected to the gas storage section (2), the replenishment section (4) configured to supplement gas to the gas storage section (2). System may moreover include a pressure control unit (5) connected to the gas storage section (2), configured to stabilize a pressure inside the gas storage section (2). The therapeutic gas delivery apparatus may also include a flow control unit (6) coupled to the gas output section (3), configured to control a quantity of the therapeutic gas delivered through the gas output section (3).

Implementations of the therapeutic gas delivery apparatus may include one or more of the following features. The pressure control unit (5) is configured to stabilize the pressure inside the gas storage section (2) at a preset value greater than 120 centimeters of water column. The gas output section is configured to deliver the therapeutic gas to a breathing apparatus, and the pressure control unit (5) is configured to keep the pressure inside the gas storage section (2) higher than a pressure in an inspiratory branch of the breathing apparatus. When a patient downstream of the gas output section (3) is in an exhalation phase or a flow rate of the therapeutic gas output to the patient by the gas output section (3) is less than a flow rate of the therapeutic gas provided by the therapeutic gas source (1), at least a portion of the therapeutic gas provided by the therapeutic gas source (1) is stored into the gas storage section (2).

When the flow rate of the therapeutic gas output to the patient by the gas output section (3) exceeds the flow rate provided by the therapeutic gas source (1), the therapeutic gas stored in the gas storage section (2) is spontaneously output to the gas output section (3).

The gas supplemented by the replenishment section (4) may include air or the therapeutic gas. An inlet of the replenishment section (4) is connected to a power source, ensuring a driving force to supplement the gas into the gas storage section (2), where the power source may include a high-pressure gas cylinder, a central gas source in a hospital, or a gas pump.

When the replenishment section (4) is connected to a power source, the pressure control unit (5) may include a backpressure valve (5a), where the backpressure valve (5a) is configured to: when the pressure inside the gas storage section (2) exceeds a preset value, release gas in the gas storage section (2) through a relief port of the backpressure valve (5a) to stabilize the pressure inside the gas storage section (2).

When the power source connected to the replenishment section (4) may include a high-pressure gas cylinder or gas pump, the pressure control unit (5) may include a combination of a pressure-reducing valve (5b) and the backpressure valve (5a), where: the pressure-reducing valve (5b) is configured to stabilize an input pressure from the power source.

The pressure control unit (5) may include a valve component that provides both pressure-reducing function and backpressure function. The pressure control unit (5) may include a first mass flow controller (MFC) further coupled to the replenishment section (4) to control a flow rate of gas supplemented into the gas storage section (2) by the replenishment section (4). A relief air path is configured in between the first MFC and the gas storage section (2), where the relief air path may include a second MFC configured to control a flow rate of gas being released from the gas storage section (2), thereby stabilizing the pressure inside the gas storage section (2).

The pressure control unit (5) may include a combination of a pressure sensor and an electric control valve, where: the pressure sensor is configured to detect the pressure in the gas storage section (2), and the electric control valve is configured to control an opening based on the detected pressure to adjust an airflow passing through the opening. The electric control valve may include a solenoid valve or a proportional valve.

A cross-sectional area of the gas storage section (2) is between 1 mm to 4 cm, inclusive. The replenishment section (4) is further connected to the therapeutic gas source (1) and configured to supplement gas to the therapeutic gas source (1). Therapeutic gas source (1) may include an electrochemical instant preparation device for electrochemically generating nitric oxide (NO), and the replenishment section (4) is further configured to input purging gas into the therapeutic gas source (1) for purging electrodes and carrying out the electrochemically generated NO, where the purging gas may include air or nitrogen.

Therapeutic gas source (1) may include an instant preparation device for generating NO using an arc method, and the replenishment section (4) is configured to input reaction gas into the therapeutic gas source (1), where electrodes inside an reaction chamber of the therapeutic gas source 1 are used to generate NO through high-voltage electric shocks, and the generated NO is carried out by an excess portion of the reaction gas.

The reaction gas may include air or oxygen-nitrogen-containing gas. The replenishment section (4) is configured to connect to both the gas storage section (2) and the therapeutic gas source (1), and input the gas through a power source to both the gas storage section (2) and the therapeutic gas source (1). The replenishment section (4) may include a first replenishment section connected to the gas storage section (2), and a second replenishment section connected to the therapeutic gas source (1), where the first replenishment section and the second replenishment section are connected to different power sources to transport different gases into the gas storage section (2) and the therapeutic gas source (1), respectively.

The first replenishment section is configured to input air into the gas storage section (2); and the second replenishment section is configured to input nitrogen into the therapeutic gas source (1).

The therapeutic gas delivery system may further include: a second flow control unit (7) installed downstream of the therapeutic gas source (1) to control a flow rate of the therapeutic gas output from the therapeutic gas source (1). The therapeutic gas delivery system may further include: a second flow control unit (7) installed upstream of the therapeutic gas source (1) to control a flow rate of gas entering the therapeutic gas source (1).

It is to be understood that both the foregoing general description and the following detailed description are examples and explanatory only and are not restrictive of the disclosed embodiments as claimed.

The accompanying drawings constitute a part of this specification. The drawings illustrate several embodiments of the present disclosure and, together with the description, serve to explain the principles of certain disclosed embodiments as set forth in the accompanying claims.

### BRIEF DESCRIPTION OF THE DRAWINGS

FIG. 1 is a schematic diagram of a therapeutic gas delivery apparatus, according to a first embodiment of the present disclosure.

FIG. 2 is a schematic diagram of a therapeutic gas delivery apparatus, according to a second embodiment of the present disclosure.

FIG. 3 is a schematic diagram of a therapeutic gas delivery apparatus, according to a third embodiment of the present disclosure.

FIG. 4 is a schematic diagram of a therapeutic gas delivery apparatus, according to a fourth embodiment of the present disclosure.

FIG. 5 is a schematic diagram of a therapeutic gas delivery apparatus, according to a fifth embodiment of the present disclosure.

FIG. 6 is a schematic diagram of an electrochemical instant preparation device, according to some embodiments of the present disclosure.

FIG. 7 is a schematic diagram of an arc method instant preparation device, according to some embodiments of the present disclosure.

FIG. 8 is a schematic diagram of a Nitric Oxide (NO) supply module in a respiratory device, according to a first embodiment of the present disclosure.

FIG. 9 is a schematic diagram of the NO supply module in the respiratory device, according to a second embodiment of the present disclosure.

FIG. 10 is a schematic diagram of the NO supply module in the respiratory device, according to a second embodiment of the present disclosure.

FIG. 11 is a schematic diagram of the NO supply module in the respiratory device, according to a third embodiment of the present disclosure.

FIG. 12 is a schematic diagram of the NO supply module in the respiratory device, according to a fourth embodiment of the present disclosure.

FIG. 13 is a schematic diagram of the NO supply module in the respiratory device, according to a fifth embodiment of the present disclosure.

FIG. 14 is a schematic diagram of the NO supply module in the respiratory device, according to a sixth embodiment of the present disclosure.

FIG. 15 is a schematic diagram of the NO supply module in the respiratory device, according to a seventh embodiment of the present disclosure.

FIG. 16 is a schematic diagram of the NO supply module installed in the respiratory device through a mounting slot, according to some embodiments of the present disclosure.

FIG. 17 is a schematic diagram of NO preparation and transmission system, according to some embodiments of the present disclosure.

FIG. 18 is an schematic diagram of the gas storage section in the embodiments of the present disclosure.

FIG. 19 is another schematic diagram of the gas storage section in the embodiments of the present disclosure.

FIG. 20 is yet another schematic diagram of the gas storage section in the embodiments of the present disclosure.

### DETAILED DESCRIPTION

Reference will now be made in detail to disclosed embodiments. Unless otherwise defined, technical or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus, the materials, methods, and examples are illustrative only and are not intended to be necessarily limiting.

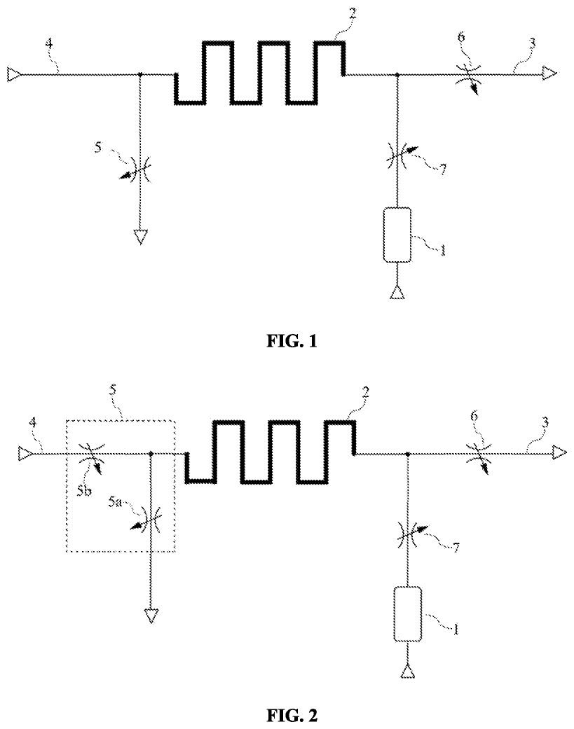

FIG. 1 is a schematic diagram of a therapeutic gas delivery apparatus, according to a first embodiment of the present disclosure. As shown, the therapeutic gas delivery apparatus includes a therapeutic gas source (1), a gas storage section (2), a gas output section (3), a replenishment section (4), a pressure control unit (5), and a flow control unit (6).

The therapeutic gas source (1) is used for the instant generation of therapeutic gases, such as NO, CO, H₂S, H₂, etc.

The gas storage section (2) is connected downstream of the therapeutic gas source (1) and configured to store therapeutic gas supplied by the therapeutic gas source (1).

The gas output section (3) is connected downstream of the therapeutic gas source (1), allowing the therapeutic gas to be transmitted to the patient.

The replenishment section (4) is connected to the gas storage section (2), through which gas can be supplemented into the gas storage section (2).

The pressure control unit (5) is connected to the gas storage section (2), and configured to stabilize the pressure inside the gas storage section (2) at a preset value. In some embodiments, the preset value is generally greater than 120 centimeters of water column. By stabilizing the pressure inside the gas storage section (2) at the preset value, the pressure inside the gas storage section (2) is ensured to be higher than the pressure in the inspiratory branch of a ventilator, allowing the therapeutic gas inside the gas storage section (2) to spontaneously output to the ventilator pipeline. The higher the preset pressure value, the greater the maximum flow rate that the therapeutic gas delivery apparatus can achieve, leading to an improved the breath-following effect. However, an increase in the pressure value also raises the reliability requirements for the apparatus.

The flow control unit (6) is located on or upstream of the gas output section (3) and configured to control the quantity or flow rate of therapeutic gas being delivered to the patient.

When the patient downstream of the gas output section (3) is in the exhalation phase or the amount of gas transmitted to the patient's end by the gas output section (3) is less than the amount provided by the therapeutic gas source (1), the excess therapeutic gas enters the gas storage section (2), and the gas storage section (2) stores all or part of the excess therapeutic gas.

When the amount of gas transmitted to the patient's end by the gas output section (3) exceeds the amount provided by the therapeutic gas source (1), the therapeutic gas stored in the gas storage section (2) spontaneously transfers to the gas output section (3). The gas supplemented by the replenishment section (4) into the gas storage section (2) can be air or therapeutic gas, etc. The inlet of the replenishment section (4) may be connected to a power source, providing sufficient driving force to supplement gas into the gas storage section (2). For example, the power source can be a high-pressure gas cylinder, a central gas source in a hospital, or a gas pump.

There are various ways to implement the pressure control unit (5). As the first embodiment illustrated in FIG. 1, when the replenishment section (4) is connected to a stable and controllable power source, the pressure control unit (5) can be a backpressure valve. When new therapeutic gas is introduced into the gas storage section (2), causing the gas pressure inside the gas storage section (2) to exceed a preset value, the gas will be vented outward from the gas storage section (2) through the relief port of the backpressure valve to maintain stable pressure within the gas storage section (2).

As a second embodiment illustrated in FIG. 2, when the power source connected to the replenishment section (4) is a high-pressure gas cylinder or gas pump, the pressure control unit (5) can include a pressure-reducing valve (5b) and a backpressure valve (5a). The pressure-reducing valve (5b) lowers and stabilizes the pressure of the input from the power source. When the gas pressure inside the gas storage section (2) exceeds a preset value, gas is vented outward from the gas storage section (2) through the relief port of the backpressure valve (5a) to maintain stable pressure within the gas storage section (2).

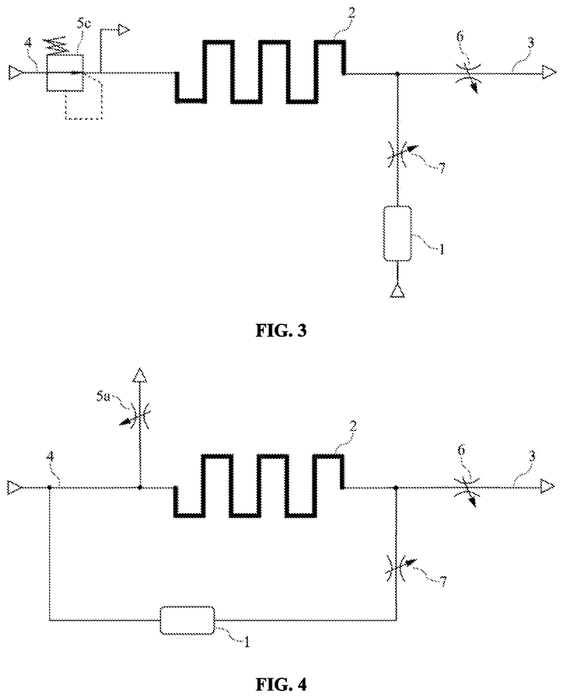

As a third embodiment illustrated in FIG. 3, some valve devices/components may have both pressure-reducing and backpressure functions. Therefore, such a valve component (5c) can be installed on the gas storage section (4), achieving the same effect as the pressure-reducing valve (5b) and the backpressure valve (5a) in the second embodiment.

In addition to the aforementioned mechanical valve forms, the pressure control unit can also take forms such as a Mass Flow Controller (MFC). For example, a first mass flow controller may be placed on the replenishment section (4) to control the mass flow rate of gas supplemented into the gas storage section (2). A relief air path may be configured between the first mass flow controller and the gas storage section (2), with a second mass flow controller arranged on the relief air path to control the mass flow rate of gas being vented, thereby achieving the effect of maintaining stable pressure within the gas storage section (2).

In some embodiments, the pressure control unit (5) can also be a combination of a pressure sensor and an electric control valve, such as a solenoid valve, proportional valve, etc. The pressure sensor is used to detect the pressure in the gas storage section (2), and the electric control valve adjusts its opening based on the detected pressure value to control the size of the airflow passing through. For example, if a low pressure is detected, then the airflow passing through is reduced. The pressure sensor can also be located downstream of the gas storage section (2), but the delay in detecting pressure changes due to flow adjustments made by the electric control valve may be less conducive to pressure control.

In some embodiments, the pressure control unit (5) can be installed on the replenishment section (4), or on the gas storage section (2). It is generally not recommended to place the pressure control unit (5) near the gas output section (3) because when excess therapeutic gas is introduced into the gas storage section (2), the pressure control unit (5) will vent some of the older gas from the gas storage section (2) to make room for the newly introduced therapeutic gas. If the placement of the pressure control unit (5) is too close to the gas output section (3), it will reduce the actual volume of the gas storage section (2) available for storing therapeutic gas. To maximize the usable volume of the gas storage section (2) and minimize the size of the gas storage section (2), it is preferred to install the pressure control unit (5) on the replenishment section (4).

In some embodiments, a cross-sectional area S of the gas storage section (2) ranges from 1 mm² to 4 cm², inclusive. A cross-sectional area S that is close to or below the lower limit will lead to increased air resistance, making it difficult to achieve the effect of rapid gas transmission. A cross-sectional area S that is close to or exceeds the upper limit will intensify the diffusion and mixing of the therapeutic gas with the supplemented gas at their interface, affecting the concentration of the output therapeutic gas and reducing the utilization rate of the produced therapeutic gas.

As a fourth embodiment illustrated in FIG. 4, the replenishment section (4) can also be connected to the therapeutic gas source (1) to supplement gas to the therapeutic gas source (1).

In some embodiments, when the therapeutic gas source (1) is an electrochemical instant preparation device for electrochemically generating NO, the replenishment section (4) can input purging gas (air, nitrogen, etc.) for purging the electrodes and carrying out the electrochemically generated NO gas.

An example electrochemical instant preparation device is illustrated in FIG. 6. As shown, the example electrochemical instant preparation device includes a reaction chamber (11), which has a gas area and a liquid area. The liquid area is configured to contain a reaction medium (12), and the gas area is configured to contain product gas including NO. Electrode (13) contacts the reaction medium (12), and by applying a predetermined current or voltage to electrode (13), NO gas can be produced within reaction chamber (11). A purging gas inlet (14) is used to introduce purging gas into the reaction medium (12) to sweep out the NO gas produced in the reaction medium (12), with the purging gas being air, nitrogen, etc. Furthermore, the reaction medium (12) may include a buffer solution, a nitrite ion source, and a catalyst, where the catalyst includes a metal-ligand complex, and the nitrite ion source includes one or more types of nitrites. For example, the composition of the reaction medium (12) may refer to the content disclosed in the Chinese Patent Publication number CN114318357A, published on Apr. 12, 2022, which discloses an electrolyte for achieving high concentration output of NO, and the corresponding electrolytic cell and electrolysis method. The content of Chinese Patent Publication number CN114318357A is incorporated into this application by reference. An example implementation of the electrochemical instant preparation device for generating NO may refer to the content disclosed in the Chinese patent publication number CN110831640A, published on Feb. 21, 2020, which discloses a nitric oxide generation system for a gas delivery device. The content of Chinese patent publication number CN110831640A is also incorporated into this application by reference.

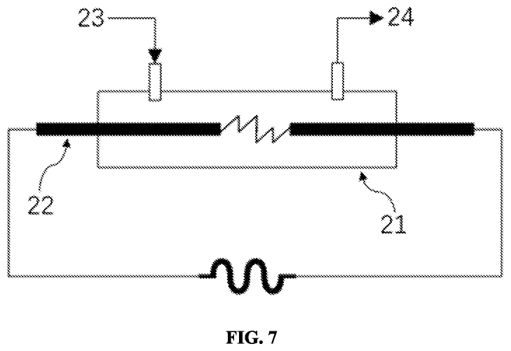

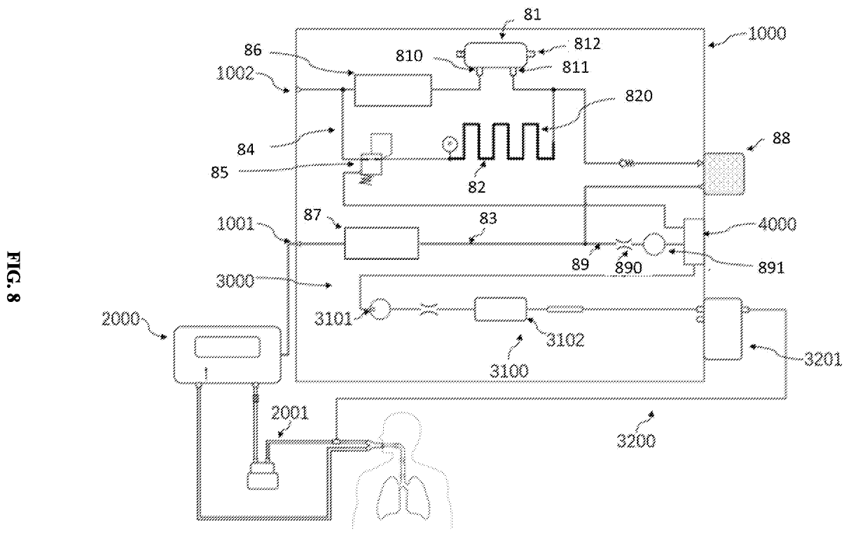

In some embodiments, when the therapeutic gas source (1) is an instant preparation device using an arc method for generating NO, the replenishment section (4) can input reaction gas (air, oxygen-nitrogen-containing gas, etc.). An example instant preparation device using an arc method is illustrated in FIG. 7. As shown in FIG. 7, the instant preparation device using the arc method may include a reaction chamber (21) that contains one or more electrodes (22). The electrodes inside the reaction chamber (21) of the therapeutic gas source (1) generate NO through high-voltage electric shocks, and the produced NO is carried out by the excess reaction gas. The instant preparation device may also include a reaction gas inlet (23) for introducing the reaction gas into reaction chamber (21). The reaction gas may be air. Electrode (22) is configured to use a high-voltage circuit to generate product gas from the reaction gas, with the product gas containing a desired amount of NO.

Referring back to FIG. 4, the replenishment section (4) can be connected to both the gas storage section (2) and the therapeutic gas source (1), inputting the same gas (such as air) through a power source to both the gas storage section (2) and the therapeutic gas source (1). Alternatively, two replenishment sections (4) can be set up, each connected to the gas storage section (2) and the therapeutic gas source (1), respectively, with each of the two replenishment sections (4) connected to different power sources to transport different gases, such as inputting air into the gas storage section (2) and nitrogen into the therapeutic gas source (1).

Additionally, a flow control unit (7) can be installed downstream of the therapeutic gas source 1 to control the flow rate of the therapeutic gas output from the therapeutic gas source 1.

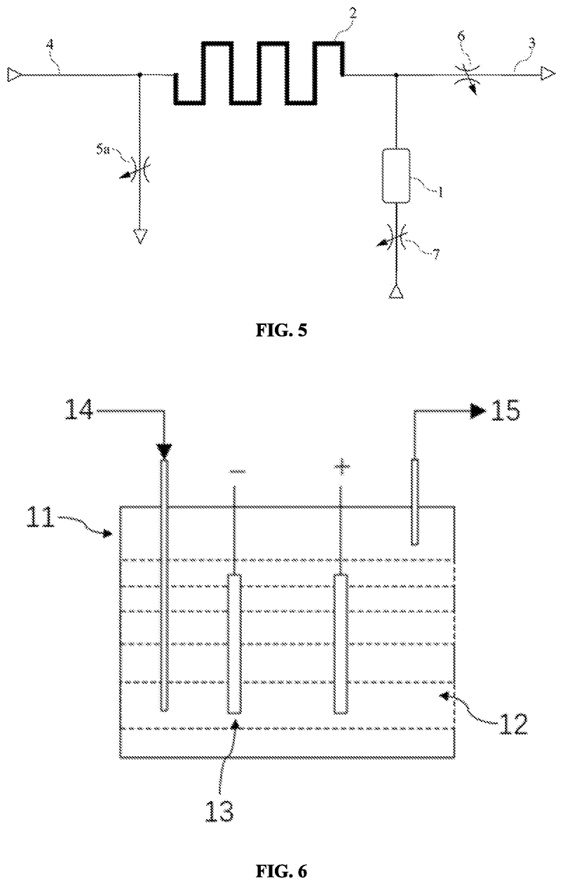

The flow control unit (7) can also be installed upstream of the therapeutic gas source (1), as shown in the fifth embodiment illustrated in FIG. 5, to control the gas flow rate entering the therapeutic gas source 1.

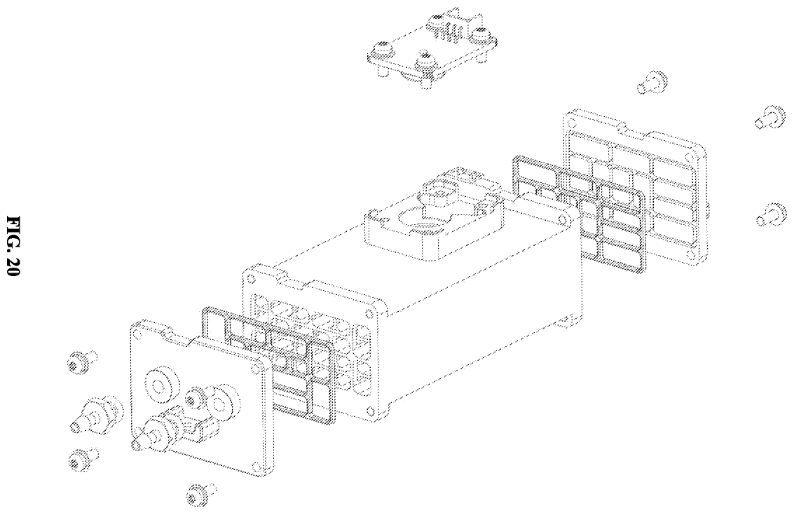

The therapeutic gas delivery apparatus described above and illustrated in FIGS. 1-7 is distinguishable from existing NO generating and delivering systems and methods from several aspects. For instance, Chinese Patent Publication No. CN110573454B describes a system and method for generating NO (e.g., paragraph [0227] and FIGS. 19-25 of the specification). The structures mentioned in CN110573454B include buffer tanks, pistons, diaphragms, and diaphragm drivers, forming a temporary storage and the NO gas is output through these structures. The disclosed solution in CN110573454B requires a certain volume of buffer tank to store NO gas, which faces limitations on miniaturizing the device. In addition, the output of NO gas relies on mechanical structures such as pistons and diaphragms, which can experience friction and wear during their operation. The action of pistons and diaphragms needs to be controlled through signal transmission, presenting challenges to the immediacy and reliability of system operation.

The above-identified limitations of CN110573454B are addressed by the technical solution described in this disclosure. For instance, the power source for driving the output of NO gas from the gas storage section (2) is the stable pressure within the gas storage section (2). The medium for driving the output of NO gas is the supplementary gas filled into the gas storage section (2) through the replenishment section (4) (the interface between the supplementary gas and the NO gas inside the gas storage section (2) can be approximately considered as a piston). During the output process of NO gas inside the gas storage section (2), there is no need for signal transmission or other forms of control. The output can be instantaneously achieved through pressure reliance. Moreover, using the supplementary gas as the medium during the output process eliminates friction and wear.

In summary, the therapeutic gas delivery apparatus described herein has at least the following technical benefits. Firstly, it is capable of achieving breath-following gas output with a sufficiently small device size. The miniaturization and lightweight design significantly reduce the limitations imposed by therapeutic settings, facilitating easier integration with other treatment devices.

Furthermore, due to the small size of the gas storage section, there is minimal accumulation of NO gas over long periods, which in turn minimizes the production of NO₂ gas within the gas storage section.

Additionally, the therapeutic gas delivery apparatus described herein does not require complex electromagnetic components or signal transmission for control coordination, eliminating the presence of wear parts. This aspect contributes to the high reliability and immediacy of the apparatus.

Another notable advantage is the efficient utilization of the therapeutic gas generated by the apparatus. Compared to the “gas storage tank” technology mentioned in the background art and in Chinese Patent Publication No. CN110573454B, the apparatus described in this disclosure demonstrates a shorter rise time when outputting therapeutic gas. In systems with a gas storage tank, the initial phase involves mixing NO gas with air until an even mixture is achieved before the output concentration of NO can stabilize. In contrast, the apparatus described herein uses a gas storage section-based system with a smaller diameter and volume. This design allows for a quick expulsion of the original gas inside the storage section upon the introduction of NO gas, resulting in a rapid adjustment to the desired NO concentration within one to two breathing cycles. This leads to a shorter concentration rise period and a faster response speed, highlighting the efficiency and responsiveness of the described therapeutic gas delivery apparatus.

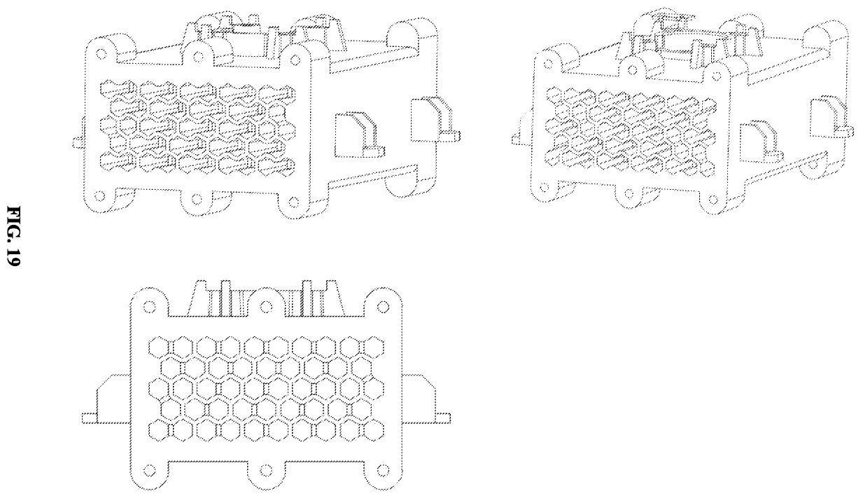

The therapeutic gas delivery apparatus described in FIGS. 1-7 may be incorporated into a respiratory device or a system as a nitric oxide (NO) supply module. The following description illustrate an example respiratory device (FIGS. 8-16) and an example respiratory system (FIG. 17).

### A Respiratory Device with a Nitric Oxide (NO) Supply Module

As mentioned in the background section, breath-following therapeutic gas output requires the ability to rapidly inject a certain flow rate of therapeutic gas based on the respiratory rate, flow, and pressure of devices such as ventilators and anesthesia machines. In some cases, the flow rate may need to reach over 120 L/min in a short period.

To achieve the breath-following transmission, a gas storage container may be configured upstream of the therapeutic gas input pipeline with a certain pressure and capacity. This gas storage container stores therapeutic gas at the appropriate times and releases the stored therapeutic gas when the inhalation flow rate increases rapidly in a short period, compensating for any shortfall in the instant preparation and transmission flow rate of the therapeutic gas unit.

Existing NO therapy devices that implement the functionality of breath-following transmission typically have a large volume and weight. When used in conjunction with respiratory devices such as ventilators, the NO therapy devices require their own dedicated space. This is disadvantageous in environments like the ICU, where space is highly limited. When used standalone, the volume and weight constraints of the existing NO therapy devices also make them difficult to apply in scenarios where portability is needed, such as home use or outdoor settings.

To address the technical challenges of the existing solutions, the therapeutic gas delivery apparatus described in FIGS. 1-7 may be adopted, as a nitric oxide (NO) supply module in a respiratory device.

A first embodiment of the NO supply module is illustrated in FIG. 8. As shown, the NO supply module may include a housing (1000). The housing (1000) may include a reaction chamber (81) with an inlet (810) and an outlet (811). The reaction chamber (81) may further include electrodes (812). The inlet (810) allows the reaction gas flow (typically air) to enter, and the electrodes (812) enable the reaction gas flow passing through the reaction chamber (81) to generate nitric oxide product gas, with the outlet (811) releasing the airflow containing the product gas.

The housing (1000) may further include a gas storage section (82), located downstream of the outlet (811). The gas storage section (82) is configured to store at least part of the product gas from the outlet (811) at certain times. The housing (1000) may further include a gas transmission section (83), located downstream of the outlet (811). The gas transmission section (83) is configured to export the airflow containing the product gas outside the housing (1000).

The housing (1000) may further include a power source (84) coupled to the gas storage section (82) to maintain a stable internal gas pressure within the gas storage section (82).

When the flow rate exported by the gas transmission section (83) outside the housing (1000) is less than the flow rate supplied by the outlet (811) to the gas transmission section (83), the excess gas is directed into the gas storage section (82) to store at least part of this excess gas. When the flow rate that needs to be exported by the gas transmission section (83) outside the housing (1000) exceeds the flow rate provided by the outlet (811) to the gas transmission section (83), the gas stored inside the gas storage section (82) is directed towards the gas transmission section (83).

Furthermore, the housing (1000) may be designed as a detachable assembly incorporated into a respiratory device (2000).

The housing (1000) may be equipped with a gas transmission interface (1001), which connects to the gas transmission section (83). The respiratory device (2000) may feature an interface that compatibly connects with the gas transmission interface (1001) and is linked to the inhalation branch (2001) of the respiratory device (2000).

In the first embodiment of the NO supply module illustrated in FIG. 8, the housing (1000) is equipped with an air intake interface (1002). The respiratory device (2000) features an interface that connects compatibly with the air intake interface (1002), and this interface is connected to the internal airway of the respiratory device (2000). The inlet (810) of the reaction chamber (81) is connected to the air intake interface (1002), supplying the reaction gas flow to the reaction chamber (81) through the internal airway of the respiratory device (2000). The gas storage section (82) is also connected to the air intake interface (1002), receiving gas through the internal airway of the respiratory device (2000), in which the air intake interface (1002) serves as the power source (84).

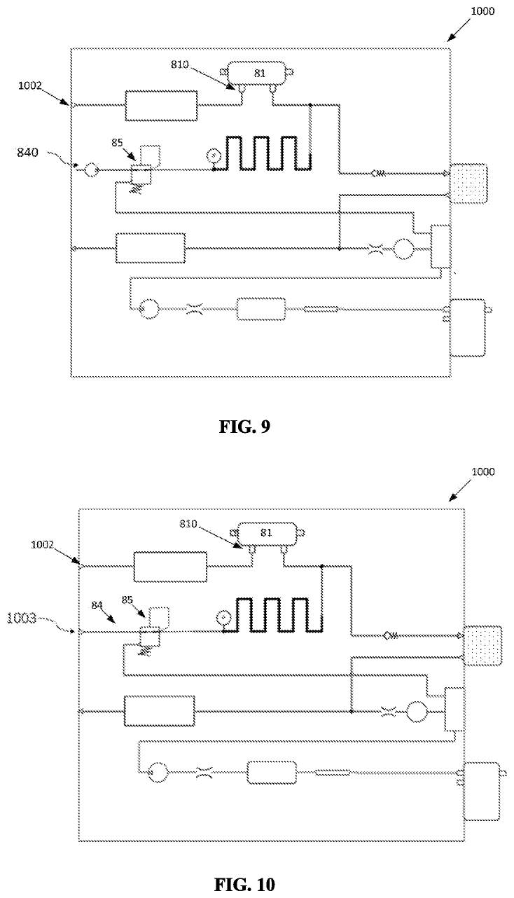

Furthermore, the power source (84) in FIG. 8 includes a pressure control device (85), which is used to maintain the stable internal gas pressure within the gas storage section (82). The pressure control device (85) can be a pressure-reducing valve with a relief function (effectively integrating the functions of a backpressure valve and a pressure-reducing valve into one unit), or it can be a combination of a pressure-reducing valve and a backpressure valve as shown in FIG. 4 of Chinese Patent Publication No. CN2023106604381, or it can be a set of cooperating mass flow controllers as shown in FIG. 9 of Chinese Patent Publication No. CN2023106604381.

FIG. 9 is a schematic diagram of the NO supply module in the respiratory device, according to a second embodiment of the present disclosure. In the second embodiment shown in FIG. 9, the housing (1000) is also equipped with an air intake interface (1002). The respiratory device (not shown in FIG. 9, see 2000 in FIG. 8) features an interface that connects compatibly with the air intake interface (1002), and this interface is connected to the internal airway of the respiratory device. The inlet (810) of reaction chamber (81) is connected to the air intake interface (1002), which supplies the reaction gas flow to the reaction chamber (81) through the internal airway of the respiratory device. The power source (84) includes a gas pump (840) located within the housing (1000). This gas pump (840) is connected to the gas storage section (82) and is used to supply gas to the gas storage section (82). Furthermore, the power source also includes a pressure control device (85), which may use the same pressure control device from the first embodiment (as shown in FIG. 8).

FIG. 10 is a schematic diagram of the NO supply module in the respiratory device, according to a third embodiment of the present disclosure. In the third embodiment shown in FIG. 10, the housing (1000) is equipped with an air intake interface (1002). The respiratory device (not shown in FIG. 9, see 2000 in FIG. 8) features an interface that matches and connects with the air intake interface (1002), and this interface is linked to the internal airway of the respiratory device. The inlet (810) of the reaction chamber (81) is connected to the air intake interface (1002), which supplies the reaction gas flow to the reaction chamber (81) through the internal airway of the respiratory device.

Additionally, the housing (1000) in FIG. 10 also has a gas supply interface (1003). The respiratory device is equipped with an interface that matches and connects with the gas supply interface (1003), and this interface is connected to the internal airway of the respiratory device. The gas storage section (82) is connected to the gas supply interface (1003), which supplies gas to the gas storage section (82) through the internal airway of the respiratory device and serves as the power source (84). Furthermore, the power source (84) may also include a pressure control device (85) from the first and second embodiments.

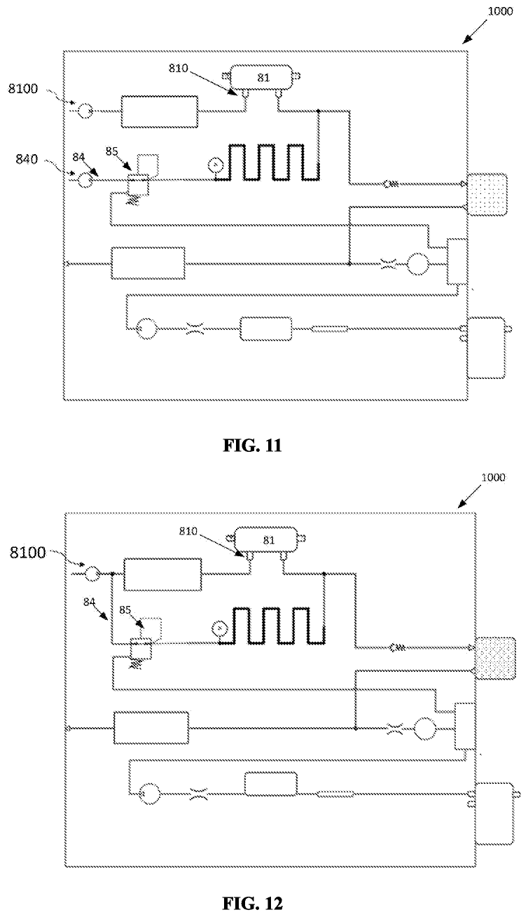

FIG. 11 is a schematic diagram of the NO supply module in the respiratory device, according to a fourth embodiment of the present disclosure. In the fourth embodiment shown in FIG. 11, the housing (1000) does not have an air intake interface (e.g., 1002 in FIGS. 8-10) or a gas supply interface (e.g., 1003 in FIGS. 8-10). Within the housing (1000), there is a first gas pump (8100), and the inlet (810) of the reaction chamber (81) is connected to this first gas pump (8100), which supplies the reaction gas flow to the reaction chamber (81). The power source (84) includes a second gas pump (840) located within the housing (1000). This second gas pump (840) is connected to the gas storage section (82), and the pump is used to supply gas to the gas storage section (82). Furthermore, the power source (84) also includes a pressure control device (85) from the previously described embodiments.

FIG. 12 is a schematic diagram of the NO supply module in the respiratory device, according to a fifth embodiment of the present disclosure. In the fifth embodiment shown in FIG. 12, the housing (1000) does not have an air intake interface (e.g., 1002 in FIGS. 8-10) or a gas supply interface (e.g., 1003 in FIGS. 8-10). Inside the housing (1000), there is a second gas pump (8100), with the inlet (810) of reaction chamber (81) connected to this second gas pump (8100), which supplies the reaction gas flow to the reaction chamber (81). The gas storage section (82) is also connected to the second gas pump (8100), which supplies gas to the gas storage section (82), thereby serving as the power source (84). Furthermore, the power source (84) includes a pressure control device (85) from the previously described embodiments.

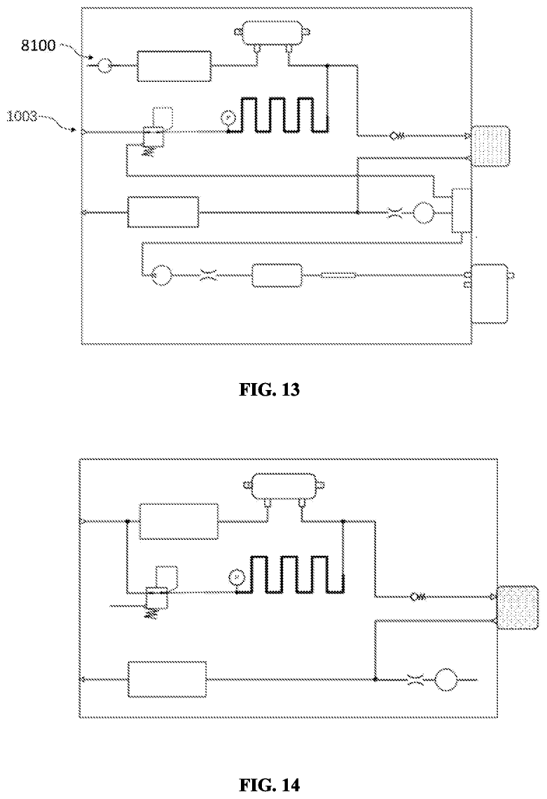

FIG. 13 is a schematic diagram of the NO supply module in the respiratory device, according to a sixth embodiment of the present disclosure. In the sixth embodiment shown in FIG. 13, the housing (1000) does not have an air intake interface (1002). Inside the housing (1000), there is a gas pump (8100), with the inlet (810) of the reaction chamber (81) connected to this gas pump (8100), which supplies the reaction gas flow to the reaction chamber (81). The housing (1000) is equipped with a gas supply interface (1003). The respiratory device (not shown in FIG. 9, see 2000 in FIG. 8) features an interface that matches and connects with the gas supply interface (1003), and this interface is connected to the internal airway of the respiratory device. The gas storage section (82) is connected to the gas supply interface (1003), which supplies gas to the gas storage section (82) through the internal airway of the respiratory device, thereby serving as the power source (84). Furthermore, the power source (84) includes a pressure control device (85) from the previously described embodiments.

In the embodiments described in disclosure, the gas storage section (82) may include at least one gas storage passage (820). The gas storage passage (820) may have a sufficiently small cross-sectional area to minimize the dispersion phenomenon between gases (i.e., reduce the dispersion at the interface between nitric oxide gas stored in the gas storage passage (820) and air input by the power source). The cross-sectional area S of the gas storage passage may be configured with in the range of 1 mm²<S<4 cm².

In addition to the various forms of the power source (84) mentioned in the previously described embodiments, it can also be in implemented in other ways. For example, the power source (84) may be in the form of a piston cylinder, and the gas storage section (82) is integrated into the piston cylinder. The controlling of the pressure in the gas storage section (82) is achieved by driving the piston rod to change the volume of the space in the gas storage section (82). Compared to the other forms mentioned in the previous embodiments, this piston cylinder structure has some drawbacks: 1) Volume impact-the internal space of the module housing is limited, and the form of the piston cylinder might cause an increase in the overall volume of the module; 2) Reliability—the piston rod of the piston cylinder requires frequent repetitive driving actions, which poses a challenge to its reliability; 3) Delay-the timing of driving the piston rod needs to be controlled by signal feedback, which can lead to delays, resulting in the internal pressure of the gas storage section (82) not matching the desired pressure and affecting the accuracy of nitric oxide output; 4) Noise; 5) Power consumption.

As another example, the power source (84) may be in the form of a gas bag. In this case, the gas storage section (82) may use a gas bag structure capable of expansion and contraction, functioning as the power source (84). In particular, the force to recover the deformation of the gas bag structure serves as the power source. This method also has certain drawbacks, such as fatigue and wear from the repeated deformation of the gas bag, leading to a limited service life.

In the embodiments of the NO supply module illustrated in this disclosure, a first flow control device (86) (as the example shown in FIG. 8 but applicable to all described embodiments) is installed on the upstream pipeline connected to the inlet (810) of the reaction chamber (81) to control the flow of reaction gas entering the reaction chamber (81). In some embodiments, the first flow control device (86) may be a Mass Flow Controller (MFC).

Furthermore, on the gas transmission section (83), a second flow control device (87) (as the example shown in FIG. 8 but applicable to all described embodiments) may be installed, which controls the flow rate of the outgoing gas stream. The second flow control device (87) may also be a Mass Flow Controller (MFC).

Using the embodiment illustrated in FIG. 8 as an example (applicable to all described embodiments), a filtration device (88) may be installed on the gas transmission section (83) to filter NO2 from the product gas. The filtration device (88) is detachably connected to the gas transmission section (83). The filtration device (88) has an inlet and an outlet, each connected to the gas transmission section (83).

Using the embodiment illustrated in FIG. 8 as an example (applicable to all described embodiments), the filtration device (88) is located on the exterior of the housing (1000). An interface for the inlet and outlet of the filtration device (88) to be plugged in is provided on the housing (1000), with the internal interface of the housing (1000) connecting to the gas transmission section (83). Alternatively, the filtration device (88) can be installed on the interior side of the housing (1000), close to the wall of the housing (1000), with a removable operation window provided at the corresponding position of the filtration device (88) on the housing (1000). The filtration device (88) is a consumable, and its filtering material (e.g., calcium hydroxide) may need to be replaced periodically as its use time increases. Placing the filtration device (88) on the exterior of the housing (1000) or close to the wall of the housing (1000) facilitates replacement. Preferably, the filtration device (88) is installed upstream of the second flow control device (87). Placing it upstream helps ensure the effectiveness of breath-following because the filtration device (88) has a filter chamber filled with filtering material. If the filtration device (88) is placed downstream of the second flow control device (87), the flow rate and timeliness of the therapeutic gas output through the second flow control device (87) and passing through the filter chamber may be affected, thereby impacting the effectiveness of breath-following.

Using the embodiment illustrated in FIG. 8 as an example (applicable to all described embodiments), a detection branch (89) is installed on the gas transmission section (83), with one end connected to the gas transmission section (83) and the other end open to the environment. The detection branch (89) is used to measure the concentration of nitric oxide in the gas transmission section (83). Furthermore, the detection branch (89) includes an air resistance (890) and a nitric oxide sensor (891) arranged in sequence from closer to further away from the gas transmission section (83). The air resistance may be set up to prevent a large amount of gas from escaping to the environment through the detection branch (89), allowing only a small amount of gas to pass through to the sensor.

In some embodiments, the detection branch (89) in the example shown in FIG. 8 is positioned upstream of the second flow control device (87) and downstream of the filtration device (88). Ideally, the detection branch (89) is placed as close as possible upstream of the second flow control device (87) to ensure that the NO concentration monitored is as close as possible to the actual output concentration. If the detection branch (89) is placed downstream of the second flow control device (87), it would be difficult for the therapeutic gas to enter the detection branch (89), likely resulting in the inability to detect the concentration of the therapeutic gas. Placing the detection branch (89) upstream of the filtration device (88) would result in a lower actual output concentration of NO.

In some embodiments, the NO supply module illustrated in FIGS. 8-13 may further include a sampling detection unit (3000), which includes a detection gas pathway (3100) located within the housing (1000) and a sampling gas pathway (3200) located outside the housing (1000). The detection gas pathway (3100) is connected to the sampling gas pathway (3200).

Using the embodiment illustrated in FIG. 8 as an example (applicable to all described embodiments), one end of the sampling gas pathway (3200) is connected to the inhalation branch (2001) of the respiratory device (2000), and the other end is connected to the detection gas pathway (3100) inside the housing (1000) through a water trap (3201). The water trap (3201) is a primarily intended to filter moisture from the sampling gas to prevent damage to downstream sensors or affect the detection results. The water trap (3201) needs to be removed periodically, so it is located on the exterior of the housing (1000), and the housing (1000) is equipped with a mounting base for installing the water trap (3201).

In some embodiments, one end of the detection gas pathway (3100) is connected to the water trap (3201), and the other end is open to the environment. The detection gas pathway (3100) is equipped with a sampling gas pump (3101) and a sensor unit (3102). The sampling gas pump (3101) provides the sampling power, and the sensor unit (3102) can include sensors such as a nitric oxide sensor, nitrogen dioxide sensor, oxygen sensor, etc., to detect components like NO, NO₂, O₂, etc., in the gas that the patient is about to inhale.

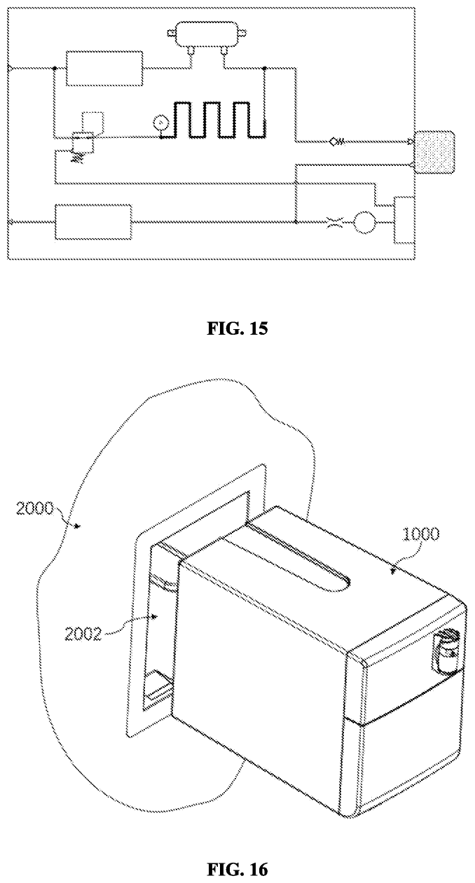

The sampling detection unit (3000) is optional in the NO supply module, as shown in the embodiments illustrated in FIGS. 14 and 15, which do not include the sampling detection unit (3000 as shown in FIG. 8). In some embodiments, the sampling detection unit (3000 as shown in FIG. 8) can be an independent module assembled with the respiratory device (2000 as shown in FIG. 8).

In some embodiments, the housing (1000) in FIG. 8-13 may be equipped with a fan (see 4000 in FIG. 8 as an example). Using the embodiment illustrated in FIG. 8 as an example (applicable to all described embodiments), the fan (4000) is located on the inner wall of the housing (1000), and ventilation holes are provided at the position of the fan (4000) on the housing (1000). The fan (4000) can also be placed on the exterior of the housing (1000), and its primary function is to facilitate cooling and ventilation.

As an example, the end of the detection branch (89) that connects to the environment is linked to the fan (4000), thereby connecting to the external environment of the housing (1000). Similarly, the end of the detection gas pathway (3100) of the sampling detection unit (3000) that connects to the environment is also linked to the fan (4000), thereby connecting to the external environment of the housing (1000). The gas escaping to the environment through the detection branch (89) may include the product gas nitric oxide, which can easily oxidize into the toxic nitrogen dioxide. If it accumulates inside the housing (1000), it could pose a safety hazard, so linking to the fan (4000) allows it to be expelled to the external environment as soon as possible. Likewise, a certain amount of nitric oxide and nitrogen dioxide in the detection gas pathway (3100) of the sampling detection unit (3000) is also expelled to the external environment as soon as possible through the fan (4000). Additionally, the fan (4000) disperses these gases before expelling them, preventing the accumulation of NO, NO2, and other exhaust gases. In some embodiment, the fan (4000) is optional within the NO supply module (see the embodiment in FIG. 14 that does not include the fan).

In some embodiments, when the pressure control device (85) is a pressure-reducing valve with a relief function, its relief port can also be connected to the fan (4000) via a pipeline. The relief port can also be directly connected to the environment. Alternatively, the relief port can be connected to the inlet of the filtration device (88) via a pipeline.

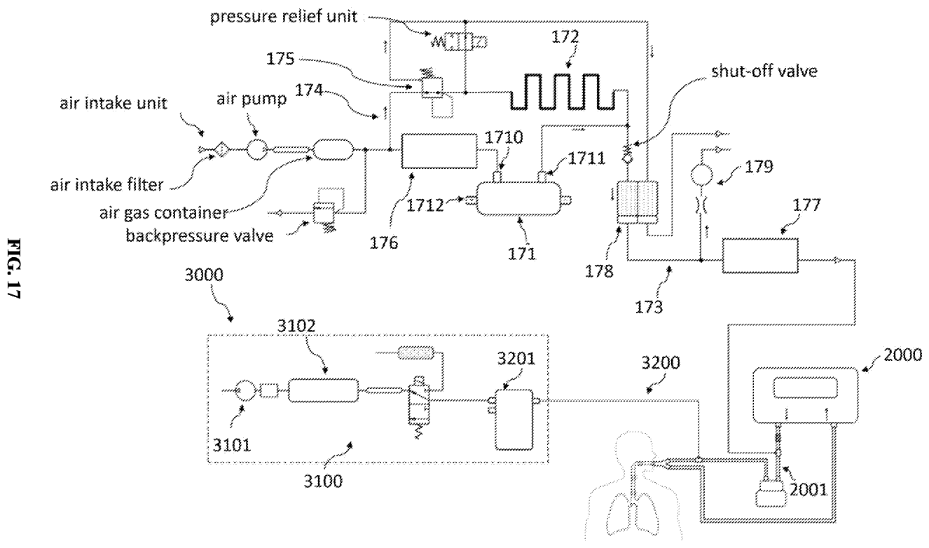

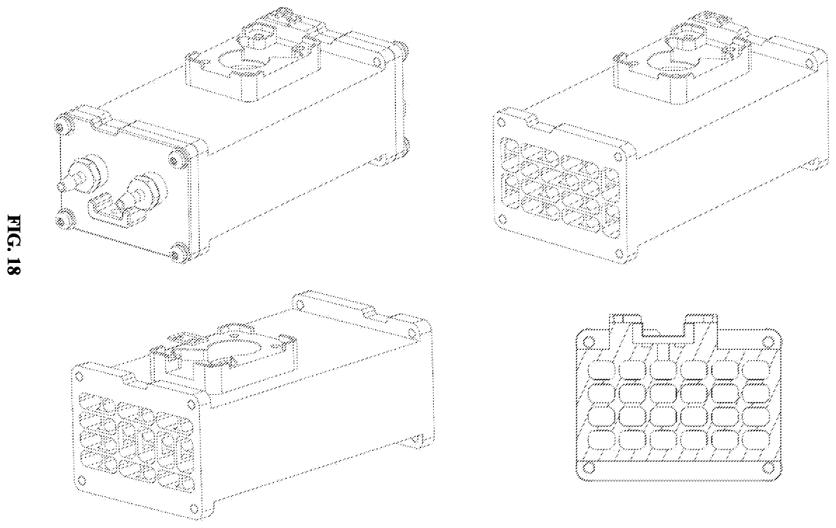

FIG. 16 is a schematic diagram of a portable NO supply device that may be installed onto a respiratory device (2000) through a mounting slot (2002), according to some embodiments of the present disclosure. The respiratory device illustrated in FIG. 16 is a portable NO supply device, in which the housing (1000) is designed to be easily portable. The mounting slot (2002) is designed to install the portable NO supply device to the respiratory apparatus (2000).

### A Nitric Oxide (NO) Preparation and Transmission System

FIG. 17 illustrates a schematic diagram of NO preparation and transmission system, according to some embodiments of the present disclosure. As shown, the NO preparation and transmission system may include an air intake unit for supplying air to the system and a reaction chamber (171), located downstream of the air intake unit. The reaction chamber may include an inlet (1710), an outlet (1711), and electrodes (1712). The inlet (1710) connects to the air intake unit to receive a reaction gas flow (e.g., air), and the electrodes (1712) enable the reaction gas flow passing through the reaction chamber (171) to generate nitric oxide product gas. The outlet (1711) releases the airflow containing the product gas.

The NO preparation and transmission system, as shown in FIG. 17, may further include a transport unit for exporting the airflow containing the product gas out of the system, the transport unit including a gas storage section (172), a gas transmission section (173), a supplementary gas section (174), and a pressure control unit (175). As shown in FIG. 17, the gas storage section (172), located downstream of the outlet (1711), is used to store at least a part of the product gas from the outlet (1711) at certain times. The gas transmission section (173), also located downstream of the outlet (1711), is used to export the airflow containing the product gas out of the system. The supplementary gas section (174), one end connected to the air intake unit and the other end connected to the gas storage section (172), is used to supplement gas to the gas storage section (172). The pressure control unit (175) is connected to the gas storage section (172) and is used to maintain the pressure inside the gas storage section (172) at a preset value. One end of the supplementary gas section (174) may also be connected to an independent air intake unit to achieve the supplementary gas function.

In some embodiments, the air intake unit may include one or more components. For instance, the air intake unit may include an air intake filter, which filters particulates, VOCs, etc., from the air to prevent damage to internal components (such as air pumps) of the device, or from being inhaled by patients. Downstream of the air intake filter, the air intake unit may further include an air pump (which can be a diaphragm pump or other types of booster pumps). This pump draws air from the environment into the device, providing an air gas source for generating NO in reaction chamber (171) and also supplying a pressurized gas source for the system.

Downstream of the air pump, the air intake unit may further include an air gas container, which aims to reduce fluctuations in the pulsating airflow produced by the air pump, stabilizing the air flow and the pressure produced by the air intake unit.

While the air pump provides a pressurized gas source, it may also produce water. If water enters the system, it could affect the production of NO therapeutic gas in the arc reaction chamber (171), as well as potentially affect the system's filters in absorbing NO₂. To address this issue, an air dehumidification unit could be added. This dehumidification unit could be placed upstream of the air pump, but water may be generated again after passing through the air pump. The dehumidification unit could be placed downstream of the air gas container, but liquid water may have already formed downstream, requiring treatment of the liquid water at a higher cost. Therefore, the dehumidification unit is preferably located between the air pump and the air gas container to quickly reduce the humidity of the compressed gas source. This dehumidification method could be a Nafion tube or other methods of filtering water or water vapor.

In some embodiments, the air intake unit may also include a backpressure valve to prevent pipe burst when the pressure of the air intake unit is too high, and a pressure sensor could be added to detect the pressure of the air intake unit. If the pressure is too high, it could stop or reduce the air extraction.

In some embodiments, when the flow rate exported by the gas transmission section (173) outside the system is less than the flow rate supplied by the outlet (1711) to the gas transmission section (173), the excess gas enters the gas storage section (172), where at least a part of this excess gas is stored. Conversely, when the flow rate that needs to be exported by the gas transmission section (173) outside the system exceeds the flow rate provided by the outlet (1711) to the gas transmission section (173), the gas stored within the gas storage section (172) is directed towards the gas transmission section (173).

Furthermore, the pressure control unit (175) can be a pressure-reducing valve with a relief function (effectively integrating the backpressure valve function and pressure-reducing valve function into one unit), or it can be a combination of a pressure-reducing valve and a backpressure valve (as shown in FIG. 4 of Chinese Patent Publication CN2023106604381), or it can be a set of cooperating mass flow controllers (as shown in FIG. 9 of Chinese Patent Publication CN2023106604381).

In some embodiments, the gas storage section (172) includes at least one gas storage passage (1720). The gas storage passage (1720) has a sufficiently small cross-sectional area to minimize the dispersion between gases (i.e., to reduce the dispersion at the interface where the nitric oxide gas stored in the gas storage passage (1720) meets the air input from the power source). The cross-sectional area of the gas storage passage is less than 20 cm², preferably less than 4 cm², and more preferably less than 1 cm². Furthermore, to fully utilize the gas storage space of the gas storage section (172) and minimize the overall space it occupies, the ports for input/output of nitric oxide gas in the gas storage section (172) are located at one end of the gas storage passage (1720), while the ports for connecting to the power source (174) are situated at the other end of the gas storage passage (1720).

In some embodiments, the air intake unit is connected to the inlet (1710) of reaction chamber (171) via a pipeline equipped with a first flow control device (176), which regulates the flow of reaction gas entering reaction chamber (171). The first flow control device (176) can also be positioned downstream of the outlet (1711) of reaction chamber (171). The first flow control device (176) may be a Mass Flow Controller (MFC).

In some embodiments, the transport unit may further include a second flow control device (177), located on the gas transmission section (3), to regulate the flow rate of the outgoing airflow. The second flow control device (177) may also be a Mass Flow Controller (MFC).

In some embodiments, the transport unit may further include a filtration device (178), positioned on the gas transmission section (173), designed to filter NO₂ from the product gas. The filtration device (178) may be detachably connected to the gas transmission section (173). The filtration device (178) may include an inlet and an outlet, each connecting to the gas transmission section (173). Moreover, the transport unit may include a detection branch (179), situated on the gas transmission section (173). One end of detection branch (179) connects to the gas transmission section (173), and the other end opens to the environment, allowing for the measurement of nitric oxide concentration in the gas transmission section (173). The detection branch (179) may include an air resistance (1790) and a nitric oxide sensor one (1791), arranged in sequence from near to far from the gas transmission section (173). The air resistance is designed to prevent a large amount of gas from escaping through detection branch (179) to the environment, allowing only a small amount of gas to pass through to the sensor.

In some embodiments, the NO preparation and transmission system in FIG. 17 may further include a sampling detection unit (3000), which may include a detection gas pathway (3100) and a sampling gas pathway (3200). The detection gas pathway (3100) is connected to the sampling gas pathway (3200). One end of the sampling gas pathway (3200) is connected to the inhalation branch (2001) of the respiratory device (2000), while the other end connects to the detection gas pathway (3100) through a water trap (3201). The water trap (3201) is designed to filter moisture from the sampled gas to prevent damage to downstream sensors or to avoid impacting the detection results.

In some embodiments, one end of the detection gas pathway (3100) connects to the water trap (3201), and the other end opens to the environment. The detection gas pathway (3100) is equipped with a sampling gas pump (3101) and a sensor unit (3102). The sampling gas pump (3101) provides the power for sampling, while the sensor unit (3102) can include sensors such as nitric oxide sensors, nitrogen dioxide sensors, oxygen sensors, etc., for detecting NO, NO₂, O₂, etc., in the gas that the patient is about to inhale.

In some embodiments, the filtration device (178) in FIG. 17 may house several independent filtration chambers to achieve different filtering functions. For example, one filtration chamber is filled with calcium hydroxide filtering agent, connected to the gas transmission section (173) for the removal of NO₂ from the gas. Another filtration chamber is filled with potassium permanganate filtering agent, used for exhaust gas removal, with its outlet connected to the environment.

When the pressure control unit (175) is a pressure-reducing valve with a relief function, its relief port can also be connected via a pipeline to the filtration device (178) chamber for exhaust gas removal. This setup prevents untreated gas from being directly released into the environment when product gas is expelled from the relief port through the filtration device (178).

In some embodiments, on the gas transmission section (173), a shut-off valve is installed upstream of the filtration device (178). When the filtration device (178) reaches the end of its life, the device doesn't need to be shut down for replacement. The filtration device (178) can be directly removed and replaced, and the shut-off valve immediately closes upon removal of the filtration device (178) to maintain stable internal pressure within the equipment. When a new filtration device (178) is reinserted, the shut-off valve is opened by the filtration device (178) to reconnect the gas transmission section (173) and the filtration device (178), allowing the NO therapeutic gas to be normally output. This shut-off valve could also be a solenoid valve.

In some embodiments, the NO preparation and transmission system in FIG. 17 may further include a pressure relief unit. The pressure relief unit may include a pressure relief pipeline. One end of this pipeline can be connected between the gas storage section (172) and the pressure control unit (175), or between the gas storage section (172) and the shut-off valve, or between the filtration device (178) and the flow control device one (176). For example, a solenoid valve may be installed on the pressure relief pipeline. When the device stops outputting NO, opening this solenoid valve allows for the rapid expulsion of NO from the system, preventing NO from remaining in the system for extended periods where it could oxidize to NO2. This also helps to equalize the internal and external pressures of the system, extending the device's lifespan. An alternative approach is to open the solenoid valve and simultaneously stop the arc after stopping NO output, using the air from the air intake unit to purge the NO gas from the system. When NO output is stopped, the gas pathway inside the system contains air. The other end of the pressure relief pipeline may be connected to the chamber of the filtration device (178) used for removing exhaust gas.

In summary, the NO preparation and transmission system in FIG. 17 provides at least the following technical advantages over existing solutions:
1) Internal integrated gas source, not dependent on external gas sources, making the device versatile for various usage scenarios, convenient for device connection, handling, or in transfer situations;
2) Internal integrated gas source processes the humidity of the incoming air, reducing the impact of moisture on the efficiency of NO production in the arc reaction chamber;
3) Internal integrated gas source processes the humidity of the incoming air, reducing the generation of more impurity gases after water vapor enters the arc reaction chamber;
4) Internal integrated gas source processes the humidity of the incoming air, reducing the possibility of water vapor condensing within the system, preventing corrosion of the system by acidic liquids formed when NO2 dissolves in water;
5) Internal integrated gas source processes the humidity of the incoming air, reducing the impact of water vapor on the filtering material's effectiveness in filtering impurity gases after entering the filter;
6) Releasing the system's internal pressure after the device is shut down to balance the internal and external pressures, extending the life of the device;
7) Purging the device after stopping treatment, ensuring the device's internal gas pathways are filled with air, to prevent NO from staying in the system and oxidizing to NO2, affecting patients in subsequent treatments;
8) The shut-off valve allows for the filter to be replaced without stopping the device, without depressurizing it, and without leaking NO treatment gas, reducing the time treatment is interrupted by filter replacement.

In the design of high-performance and reliable gas storage systems, numerous technical challenges arise. A crucial requirement for the gas storage compartment is to keep the cross-sectional area of its cavity as uniform as possible. Maintaining a minimal cross-sectional area is crucial for minimizing the mixing and dispersion of fluids between adjacent sections within the container. Traditional approaches, such as the use of long pipes and similar pipeline configurations, tend to demand significant installation space. While strategies like winding and stacking can help optimize space utilization, they fall short when dealing with special fluids, including corrosive gases or liquids like NO, NO2, and nitric acid. For these substances, conventional hose materials are inadequate, necessitating the use of materials such as PTFE and other high-fluorine substances. However, the inherent rigidity of these materials presents its own set of challenges, particularly in contexts with strict spatial constraints for container installation. This rigidity limits the feasibility of compact winding or stacking, increasing the risk of pipeline damage and the formation of dead zones within the cavity. Consequently, there is a pressing need for a gas storage solution that is compact, offers a high volume-to-space ratio, and effectively mitigates the mixing and dispersion of fluids in adjacent internal sections.

FIGS. 18 and 19 illustrate schematic diagrams of the gas storage section in the embodiments of the present disclosure. The designs in FIGS. 18 and 19 include a compact structure with a high effective-volume ratio, corrosion-resistant materials, and minimal internal fluid mixing. The structural design is to keep the interior fluid mixing and dispersion to a minimum by employing a series of continuous pipelines that maintain a uniform cross-section throughout. To optimize the volume ratio, these pipelines have a uniform thickness, and interconnections are created between adjacent channels through small perforations in their walls. Considering practical manufacturing techniques, the aperture cross-sectional area can be made less than 1 cm² to avoid excessive fluid diffusion. Materials such as polytetrafluoroethylene (PTFE), aluminum alloys, and stainless steel can be selected, with machining or injection molding as potential manufacturing processes.

In nature, the honeycomb structure (comprising nearly hexagonal shapes that connect efficiently, maximizing wall thickness usage between each) represents the most efficient use of space. Inspired by this, a honeycomb-like structure made of corrosion-resistant metal or plastic can be manufactured, as illustrated in FIGS. 18 and 19, with partial sidewalls between adjacent holes opened to create a continuous (approximate) cross-sectional pipeline container. This structure is sealed at both ends with a mesh corrosion-resistant sealing pad and end caps, allowing for a compact volume while maintaining continuous cross-sectional pathways.

The difference between FIGS. 18 and 19 is the cross-sectional shape of the cavity body of the gas storage section. When the manufacturing process use machines, the cross-section of each channel is designed to be elliptical, optimizing compatibility with machining tools. This approach yields the cross-sectional shapes illustrated in FIG. 18. However, this design choice leads to a reduced efficiency in the use of wall thickness between adjacent pipelines.

Conversely, when utilizing injection molding techniques, the cross-section of each channel is shaped as a regular hexagon. This method ensures uniform wall thickness throughout the gas storage section's inner cavity, as depicted in FIG. 19. Such a configuration maximizes the efficiency of internal space utilization.

Considering the actual manufacturing processes and the maximization of space utilization, the gas storage section's end caps may be detachable, with each end sealed using a mesh sealing pad, as shown in FIG. 20. The sealing pads are pressed against the ribbing (i.e., the wall thickness) between channels. Gas enters from one end, flows to the other end, and then passes back through adjacent channels opened in the sidewalls in the reverse direction, creating a serpentine flow that serially connects the entire channel unidirectionally, similar to a series of uniform wall thickness pipes tightly arranged within the structure.

Depending on the actual installation method, the gas inlet and outlet can be positioned differently, but it's desirable to ensure that every channel is fully utilized. The openings between adjacent channels on the sidewalls should be as close to the ends as possible to minimize dead spaces and prevent gas from lingering in these areas. The size of these openings should neither be too large nor too small, ideally matching the cross-sectional area of the channels. Too large an opening can cause fluid to disperse and mix when passing through, while too small an opening can create excessive resistance, affecting the normal flow of the gas. Apart from the channels connecting the gas inlet and outlet, the sidewalls at both ends of the remaining channels will be opened to an adjacent channel, but not to the same adjacent channel at both ends. A channel close to the container's sidewall may have an opening to the outside for a sensor installation, allowing the monitoring of internal pressure, temperature, and other conditions through the sensor installed at the opening.

The foregoing descriptions have been presented for purposes of illustration. They are not exhaustive and are not limited to precise forms or embodiments disclosed. Modifications and adaptations of the embodiments will be apparent from consideration of the specification and practice of the disclosed embodiments. For example, the described implementations include hardware, but systems and methods consistent with the present disclosure can be implemented with hardware and software. In addition, while certain components have been described as being connected to one another, such components may be integrated with one another or distributed in any suitable fashion.

The designs of the gas storage section illustrated in FIGS. 18 and 19 have several technical advantages. First, these structural designs significantly enhance the efficiency of gas storage by downsizing gas container volumes. These designs achieve this by maximizing pipeline volume within a compact footprint, addressing a key challenge in the design of gas containers. Moreover, the structures offer a substantial reduction in the volume occupied by traditional pipeline coil methods, facilitating easy installation and secure fixation of gas containers.

In addition, with corrosion resistance ensured, these structures offer more choices in materials and design, making them easier to produce and manufacture. Despite the compact size, the structures do not compromise on performance. It maintains key advantages such as preventing the mixing and dispersion of gases, ensuring the integrity and efficiency of gas utilization.

Particularly noteworthy is the application of these structures in NO therapy gas delivery devices. They adeptly manage the storage and release of NO gas to address the varying demands during the respiratory cycle of a user. During exhalation, these structures temporarily store excess NO gas, and during inhalation, when the demand for NO increases, they release the stored gas. This mechanism helps balance the real-time production and consumption of NO gas, preventing the mixing and dispersion with the driving gas or its oxidation to NO₂. Consequently, these structures enhance the safety and efficacy of nitric oxide therapy devices, broadening their scope of application.

In practical settings, the therapeutic gas delivery devices described in this document are utilized to administer NO therapy gas into a ventilator. The gas is mixed prior to patient inhalation. For example, a patient with a tidal volume of 500 ml who requires an inhalation concentration of 10ppm of NO would ideally receive a 1:9 mixture of NO gas to ventilator air. The therapeutic gas delivery device, therefore, would need to emit 50 ml of NO gas at 100 ppm concentration. A 35 ml gas storage section within the therapeutic gas delivery device can accomplish this when maintained at an internal pressure of 0.5 bar (Gauge pressure), given that the pressure in the ventilator's inspiratory limb is comparatively lower and can be disregarded. Alternatively, a 25 ml gas storage section within the therapeutic gas delivery device can accomplish the same goal when maintained at an internal pressure of 1 bar. This scenario highlights the device's compact and lightweight nature, significantly enhancing its compatibility with ventilators and other medical equipment.

If the mixing ratio is too high in the example above, the ventilator's oxygen might be undesirably diluted. On the other hand, a lower mixing ratio leads to less NO gas being released, which allows for a smaller gas storage section but necessitates a higher NO gas concentration from the device. This demands a more sophisticated production method, especially if using electric arc techniques, which can increase NO₂ production. Thus, there is an optimal range for mixing NO gas with air in the therapeutic gas delivery device.

Another key point is the relationship between pressure and volume within the gas storage section: higher pressure means a smaller required volume, but also imposes greater demands on the system's sealing and reliability.

Moreover, a patient's tidal volume directly affects the required size of the gas storage section. Some individuals may have tidal volumes exceeding 1000 milliliter (ml), or even 1500 ml. In preferred embodiments, the volume of the gas storage section is designed to be less than the patient's tidal volume. Adjusting the mixing ratio, storage section pressure, and NO concentration can help minimize the volume needed for the gas storage section.

Empirical testing suggests that, in the most effective models, the gas storage section should have a volume of less than 1500 ml; ideally less than 1200 ml; more ideally less than 1000 ml; and more ideally less than 800 ml. In certain cases, a volume less than 200 ml is sufficient. The preferred cross-sectional area of the gas storage section ranges between 1 mm² and 4 cm², inclusive; preferably below 2 cm², inclusive.

While illustrative embodiments have been described herein, the scope includes any and all embodiments having equivalent elements, modifications, omissions, combinations (e.g., of aspects across various embodiments), adaptations or alterations based on the present disclosure. Further, the steps of the disclosed methods can be modified in any manner, including reordering steps or inserting or deleting steps.

The features and advantages of the disclosure are apparent from the detailed specification. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

It is appreciated that the above-described embodiments can be implemented by hardware, or software (program codes), or a combination of hardware and software. If implemented by software, it may be stored in the above-described computer-readable media. The software, when executed by the processor can perform at least some of the steps of the disclosed methods.

In the foregoing specification, embodiments have been described with reference to numerous specific details that can vary from implementation to implementation. Certain adaptations and modifications of the described embodiments can be made. Other embodiments can be apparent to those skilled in the art from consideration of the specification and practice of the disclosure disclosed herein. It is intended that the specification and examples be considered as exemplary only, with a true scope and spirit of the disclosure being indicated by the following claims. It is also intended that the sequence of steps shown in figures are only for illustrative purposes and are not intended to imply all steps must be performed for any given method of operation or to be limited to any particular sequence of steps. As such, those skilled in the art can appreciate that these steps can be performed in a different order while implementing the same method. Further, the apparatuses shown in figures are illustrative only and a given apparatus or system may include a different combination of the components or modules of these apparatuses.

1. A therapeutic gas delivery system comprising:
a therapeutic gas source configured to generate therapeutic gas;
a gas storage section connected downstream of the therapeutic gas source, the gas storage section being configured to store at least part of the therapeutic gas from the therapeutic gas source;
a gas output section connected downstream of the gas storage section, the gas output section being configured to output the therapeutic gas on demand;
a replenishment section connected to the gas storage section, the replenishment section configured to supplement gas to the gas storage section; and
a flow control unit coupled to the gas output section, configured to control a quantity of the therapeutic gas delivered through the gas output section.

2. The therapeutic gas delivery system of claim 1, further comprising:
a pressure control unit connected to the gas storage section, configured to stabilize a pressure inside the gas storage section.

3. (canceled)

4. The therapeutic gas delivery system of claim 2, wherein the gas output section is configured to deliver the therapeutic gas to a breathing apparatus, and the pressure control unit is configured to keep the pressure inside the gas storage section higher than a pressure in an inspiratory branch of the breathing apparatus.

5. The therapeutic gas delivery system of claim 2, wherein:
when the replenishment section is connected to a power source, the pressure control unit comprises a backpressure valve, wherein the backpressure valve is configured to:
when the pressure inside the gas storage section exceeds a preset value, release gas in the gas storage section through a relief port of the backpressure valve to stabilize the pressure inside the gas storage section.

6. The therapeutic gas delivery system of claim 5, wherein:
when the power source connected to the replenishment section comprises a high-pressure gas cylinder or gas pump, the pressure control unit comprises a combination of a pressure-reducing valve and the backpressure valve, wherein:
the pressure-reducing valve is configured to stabilize an input pressure from the power source.

7. (canceled)

8. The therapeutic gas delivery system of claim 2, wherein:
the pressure control unit comprises a first mass flow controller further coupled to the replenishment section to control a flow rate of gas supplemented into the gas storage section by the replenishment section.

9. The therapeutic gas delivery system of claim 8, wherein:
a relief air path is configured in between the first MFC (mass flow controller) and the gas storage section, wherein the relief air path comprises a second MFC configured to control a flow rate of gas being released from the gas storage section, thereby stabilizing the pressure inside the gas storage section.

10. The therapeutic gas delivery system of claim 2, wherein:
the pressure control unit comprises a combination of a pressure sensor and an electric control valve, wherein:
the pressure sensor is configured to detect the pressure in the gas storage section, and
the electric control valve is configured to control an opening based on the detected pressure to adjust an airflow passing through the opening.

11. (canceled)

12. The therapeutic gas delivery system of claim 1, wherein:
when a patient downstream of the gas output section is in an exhalation phase or a flow rate of the therapeutic gas output to the patient by the gas output section is less than a flow rate of the therapeutic gas provided by the therapeutic gas source, at least a portion of the therapeutic gas provided by the therapeutic gas source is stored into the gas storage section.

13. The therapeutic gas delivery system of claim 12, wherein:
when the flow rate of the therapeutic gas output to the patient by the gas output section exceeds the flow rate provided by the therapeutic gas source, the therapeutic gas stored in the gas storage section is spontaneously output to the gas output section.

14. The therapeutic gas delivery system of claim 1, wherein:
the gas supplemented by the replenishment section comprises air, nitrogen, or the therapeutic gas.

15. The therapeutic gas delivery system of claim 1, wherein:
an inlet of the replenishment section is connected to a power source, ensuring a driving force to supplement the gas into the gas storage section, wherein the power source comprises a high-pressure gas cylinder, a central gas source in a hospital, or a gas pump.

16. (canceled)

17. The therapeutic gas delivery system of claim 1, wherein:
the replenishment section is further connected to the therapeutic gas source and configured to supplement gas to the therapeutic gas source.

18. The therapeutic gas delivery system of claim 1, wherein:
therapeutic gas source comprises an electrochemical instant preparation device for electrochemically generating nitric oxide, and
the replenishment section is further configured to input purging gas into the therapeutic gas source for purging electrodes and carrying out the electrochemically generated NO, wherein the purging gas comprises air or nitrogen.

19. The therapeutic gas delivery system of claim 1, wherein:
the therapeutic gas source comprises an instant preparation device for generating NO using an arc method, and
the replenishment section is configured to input reaction gas into the therapeutic gas source, wherein electrodes inside a reaction chamber of the therapeutic gas source are used to generate NO through high-voltage electric shocks, and the generated NO is carried out by an excess portion of the reaction gas.

20. (canceled)

21. The therapeutic gas delivery system of claim 1, wherein:
the replenishment section is configured to connect to both the gas storage section and the therapeutic gas source, and input the gas through a power source to both the gas storage section and the therapeutic gas source.

22. The therapeutic gas delivery system of claim 1, wherein:
the replenishment section comprises a first replenishment section connected to the gas storage section, and a second replenishment section connected to the therapeutic gas source, wherein the first replenishment section and the second replenishment section are connected to different power sources to transport different gases into the gas storage section and the therapeutic gas source, respectively.

23. The therapeutic gas delivery system of claim 22, wherein:
the first replenishment section is configured to input air into the gas storage section; and
the second replenishment section is configured to input nitrogen into the therapeutic gas source.

24. The therapeutic gas delivery system of claim 1, further comprising:
a second flow control unit installed downstream of the therapeutic gas source to control a flow rate of the therapeutic gas output from the therapeutic gas source, or
a second flow control unit installed upstream of the therapeutic gas source to control a flow rate of gas entering the therapeutic gas source.

25. (canceled)

26. The therapeutic gas delivery system of claim 1, wherein the therapeutic gas comprises one or more of NO, CO, H₂S, or H₂.
